(19) United States
(12) Patent Application Publication
(10) Pub. No.: US 2003/0032880 A1
(43) Pub. Date: Feb. 13, 2003

(54) Apparatus and method for ultrasonically identifying vulnerable plaque

(76) Inventor: Pauliina Moore, Palo Alto, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/171,807

(22) Filed: Jun. 13, 2002

Related U.S. Application Data
(60) Provisional application No. 60/298,235, filed on Jun. 13, 2001.

(51) Int. Cl. A61B 8/14

(52) U.S. Cl. 600/437

(57) ABSTRACT

A method of ultrasonically identifying vulnerable plaque includes gathering an intra-vascular ultrasound data signal. The intra-vascular ultrasound data signal is characterized as a function of relative amplitude and frequency to define a spectral slope associated with fibrotic tissue. Alternately, the intra-vascular ultrasound data signal is characterized as a mean power signal. Vulnerable plaque is then identified based upon the spectral slope and/or the mean power signal.

### BRIEF DESCRIPTION OF THE INVENTION

This invention relates generally to the analysis of cardiovascular activity. More particularly, this invention relates to a technique for the identification of vulnerable plaque and its risk for rupture in peripheral and coronary arteries.

### BACKGROUND OF THE INVENTION

Coronary heart disease remains the most common cause of death in developed countries and acute coronary syndrome including angina, non-Q-wave myocardial infarction (MI), Q-wave MI, and many cases of sudden cardiac death exact a considerable price on society in terms of mortality, morbidity, and health care costs, see, Fischer, et al., “Thrombosis and Coagulation Abnormalities in the Acute Coronary Syndromes,” Cardio Clin, 17(2): 283-294, 1999. Cerebrovascular stroke remains the third leading cause of medically related deaths and the second most frequent cause of neurologic morbidity in developed countries.

For patients with acute coronary syndromes, careful pathologic studies have implicated vulnerable plaque. The features that define vulnerable plaque include: 1) a thin fibrous cap with macrophage infiltration, 2) a large necrotic core containing crystals of unesterified (free) cholesterol and cholesterol esters, 3) intraplaque neovascularization, and 4) hemorrhage into a plaque, see, Burke, et al., “Coronary Risk Factors and Plaque Morphology in Men with Coronary Disease who Died Suddenly,” New England Journal of Medicine, 336-1276-82, 1997; Burke, et al., “Effect of Hypertension and Cardiac Hypertrophy in Sudden Cardiac Death,” Circulation 94, 3138-45, 1996; and Falk, et al., “Coronary Plaque Disruption,” Circulation 92, 657-71, 1995.

There are no known techniques to accurately characterize vulnerable plaque. However, there are several commercially available ultrasound-based techniques to roughly characterize tissue structure. These techniques include transdermal and intravascular sonography, which are used to diagnose, for example, possible tumors, abnormal tissue growth and structures. Intravascular ultrasound (IVUS) is mainly used to identify the amount of the narrowing of a diseased artery and possible complications of interventional procedures like vessel wall dissections. The current accuracy of IVUS is, however, limited with respect to determining the morphology of the atherosclerotic tissue to the identification of calcified tissue. Commercially available IVUS analyses provide between 30-60% accuracy in identifying other components of the vessel wall. These analyses are subjective and very much dependent on the experience of the interpreter.

Commercially available signal analysis products are designed to identify the borders of a vessel, not the components of atherorsclerotic disease and vulnerable plaque. One of the shortcomings of current ultrasound techniques is the data degradation inherent in a conventional signal path. In a conventional signal path, the original signal is amplified in a non-linear manner, is compressed, and is then filtered to obtain the “video-envelope.” This process is optimized to create a visually acceptable image of the major tissue interfaces, not for the preservation of a back-scattered ultrasound signal from within the vessel wall. In addition, the production of the video-envelope precludes the use of any techniques based on the frequency-analysis of the raw signal.

Other proposed technologies for the diagnosis of the morphology of atherosclerosis and vulnerable plaque also have problems. For example, angiography grossly underestimates the presence of arterial disease. Other new technologies under development include magnetic resonance imaging (MRI) and thermal sensors that measure the temperature of the arterial wall on the premise that the inflammatory process at the root of the problem generates heat. Elastography is used to identify different plaque components with intravascular ultrasound by analyzing possible differences in the elastic features of multiple plaque structures. Optical coherence tomography (OCT), contrast agents, and near-infrared and infrared light techniques have also been proposed. Unfortunately, each of these techniques is unrefined and therefore has limited value.

In view of the foregoing, it would be highly desirable to provide a technique for identifying vulnerable plaque.

### SUMMARY OF THE INVENTION

The invention includes a method of ultrasonically identifying vulnerable plaque. The method includes gathering an intra-vascular ultrasound data signal. The intra-vascular ultrasound data signal is characterized as a function of relative amplitude and frequency to define a spectral slope associated with fibrotic tissue. Alternately, the intra-vascular vascular ultrasound data signal is characterized as a mean power signal. Vulnerable plaque is then identified based upon the spectral slope and/or the mean power signal.

### BRIEF DESCRIPTION OF THE FIGURES

The invention is more fully appreciated in connection with the following detailed description taken in conjunction with the accompanying drawings, in which:

FIG. 1 illustrates an apparatus constructed in accordance with an embodiment of the invention.

FIG. 2 is a cross-section of a coronary artery characterized in accordance with techniques of the invention.

FIG. 3 illustrates power spectrum measurements processed in accordance with an embodiment of the invention.

FIG. 4 is a cross-section of a coronary artery characterized in accordance with techniques of the invention.

FIG. 5 is a side view of an ultrasound transducer and catheter utilized in accordance with an embodiment of the invention.

FIG. 6 is an axial view of an ultrasound transducer and catheter utilized in accordance with an embodiment of the invention.

FIG. 7 illustrates exemplary data output in the form of mean power values as a function of axial position, as produced by the data-rendering module of the invention.

FIG. 8 illustrates exemplary data output in the form of mean power values as a function of scan angle, as produced by the data-rendering module of the invention.

Like reference numerals refer to corresponding parts throughout the several views of the drawings.

### DETAILED DESCRIPTION OF THE INVENTION

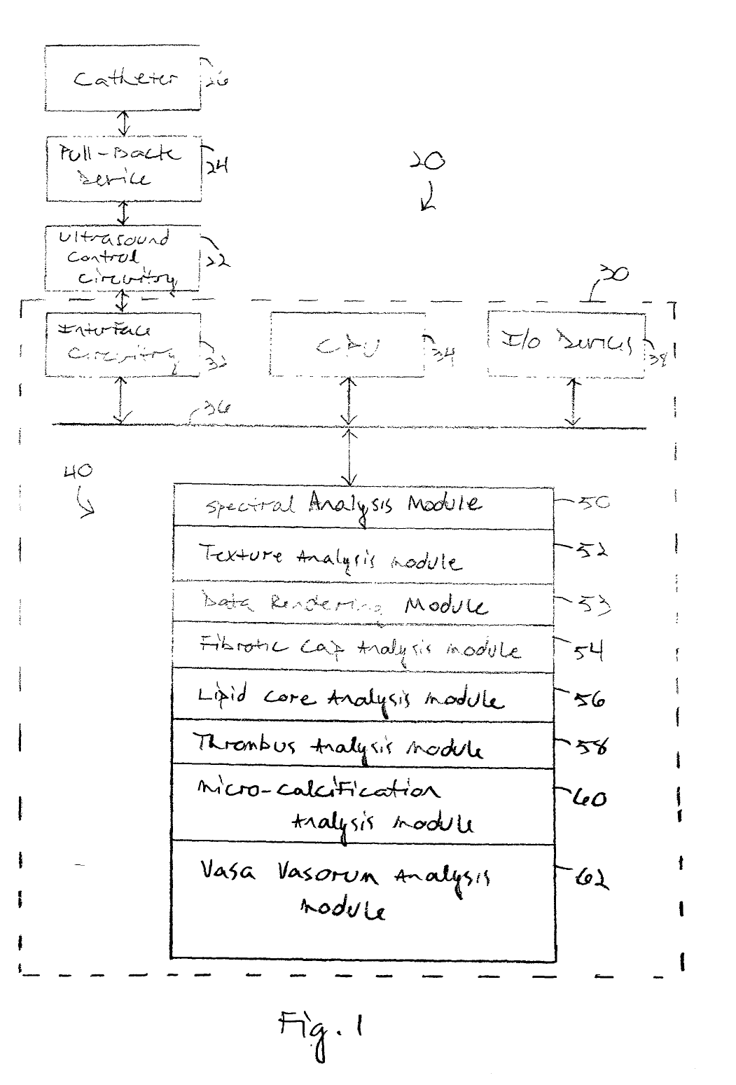

FIG. 1 illustrates an apparatus 20 constructed in accordance with an embodiment of the invention. The apparatus 20 includes ultrasound control circuitry 22 attached to a pullback device 24. A catheter 26 is connected to the pullback device 24.

A computer 30 is attached to the ultrasound control circuitry 22 through interface circuitry 32. The interface circuitry 32 is controlled by a central processing unit 34 via a system bus 36. Input/output devices 38 are also connected to the system bus 36. The input/output devices 38 may include a keyboard, mouse, video monitor, printer, and the like. Also connected to the system bus 36 is a memory 40.

The components discussed up to this point are known in the art. These components are commonly used to gather intravascular ultrasound data. The present invention is directed toward the executable programs stored in the memory 40 that are used to process the intravascular ultrasound data. In particular, unlike prior art techniques; the executable programs of the invention frequency analyze raw ultrasound signals, including back-scattered ultrasound signals.

The executable programs implement signal processing techniques performed in accordance with embodiments of the invention. The executable programs include a spectral analysis module 50, a texture analysis module 52, a data rendering module 53, a fibrotic cap analysis module 54, a lipid core analysis module 56, a thrombus analysis module 58, a micro-calcification analysis module 60, and a vasa vasorum analysis module 62. These modules are also used to automatically identify vulnerable plaque and its risk of rupture.

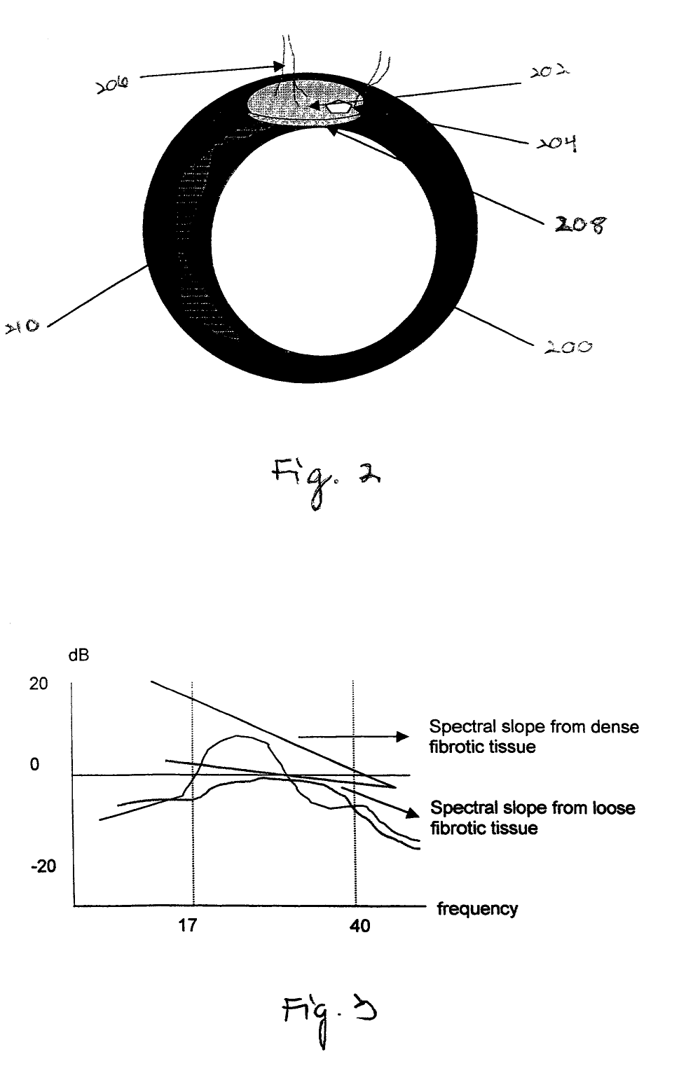

The modules may also be used to form a cross-sectional image of a coronary artery, such as shown in FIG. 2. FIG. 2 illustrates a vessel wall 200 with a lipid pool 202 formed therein. The lipid pool 202 has associated calcium 204, vasa vasorum 206, and a fibrotic cap 208. The vessel wall also has dense fibrotic tissue 210.

FIG. 2 is constructed from an ultrasound signal that is processed by the executable programs of the invention. Prior to such processing, standard ultrasound signal processing operations may be performed. For example, the ultrasound control circuitry 22 may include a real-time digitizer capable of capturing data with a wide dynamic range (e.g., up to 80 dB) at high sampling frequencies. Preferably, a minimum sampling rate of 250 MHz with 8 bits vertical resolution is used to produce high quality data at a fine temporal resolution. In one embodiment of the invention, data is captured from a complete 360 degree scan of 240 lines to a depth of 6-8 mm along each transmitted ultrasound beam, or if needed, data-collection can be limited to a chosen sector permitting higher digitization rates. The size of the region of interest will define the number of data samples along the line section of interest (e.g., length, minimum of 0.2 mm) and the number of adjacent lines from which data is collected (e.g., width, minimum of 70).

The extraction of the data points may be facilitated through the use of additional signal processing techniques, such as Fast Fourier Transforms (FFTs). The power spectra calculated from the FFT transformed vectors may be summed to obtain the average spectrum of the chosen region of interest. The size of the region of interest may be optimized to identify the frequency dependent spectral features and textural characteristics classified for vulnerable plaque and its risk for rupture, as discussed below. For relative power calculations, power spectra may be normalized with a power spectrum obtained from a number of sources. For example, the source may be a signal returning from a perfect or near-perfect specular reflector located outside the patient or in the guiding catheter. The signal may be returning from calcified plaque or from the adventitia, the outer-most region of vessel wall, which is typically highly echo-reflective, dense collagen tissue.

Data is stored with wide (e.g., up to 80 dB) total signal dynamic range and high (e.g., 40-80 dB) and low (e.g., 0-40 dB) dynamic range settings to identify features typical for vulnerable plaque. Higher dynamic range settings are used for the analysis of moderate to dense fibrotic tissue, micro-calcification and coarse calcium. Lower dynamic range settings are used for the analysis of less echo-reflective structures of vulnerable plaque, including the necrotic lipid core, vasa vasorum, and intramural or intraluminal thrombus, as discussed below.

A spectral analysis module 50 processes the data. The spectral analysis module 50 relates the relative signal amplitude as a function of frequency. The resultant spectral slope is used to characterize tissue. The spectral analysis module 50 may process parameters, such as maximum power, mean power, minimum power, y-axis intercept (intercept of the straight spectral line with the y-axis at 0 Hz), and the slope (gradient) of the power spectrum. A bandwidth that approximately corresponds to the bandwidth of the system (for 30 MHz central frequency imaging systems 17-39 MHz) is used for the analysis of the frequency dependent characteristic (intercept and slope) of the vulnerable plaque.

A texture analysis module 52 may also be used to process the ultrasound data. Preferably, the texture analysis module 52 combines features from both first and second order statistics in order to characterize the texture of the ultrasonically scanned tissue. First order statistical techniques that may be used in accordance with the invention include kurtosis, variance, and skew of the signal intensity. Second order statistical techniques that may be used in accordance with the invention include contrast, coarseness, entropy, complexity, and texture strength. Embodiments of the invention may also utilize higher order statistics, such as fractal analysis.

Data from the various signal analyses is classified according to sensitivity and specificity for the multiple features of vulnerable plaque, as discussed below. Selected subsets of parameters are assigned to identify vulnerable plaque and the relative risk for plaque rupture, as discussed below. These results are scan-converted to produce a circular image of the vessel wall and may be displayed using, for example, color encoding, to enhance the visibility and both qualitative and quantitative information of vulnerable plaque. This data, the presence, location, and size of vulnerable plaque, may also be superimposed upon traditional scan converted circular images of the vessel wall anatomy. A data rendering module 53 may be used to implement these functions.

The invention can be used to identify vulnerable plaque with a thin fibrotic cap (e.g., <100 um thick) over a necrotic lipid core. A fibrotic cap analysis module 54 may be used to identify the fibrotic cap. The fibrotic cap analysis module 54 relies upon spectral slope data from the spectral analysis module 50 to identify a fibrotic cap. Information from the spectral analysis module 50 may be used exclusively or in combination with other information to assess the risk of rupture of the fibrotic cap.

The ability to identify the thickness of the fibrotic cap depends on the axial resolution of the system, but is possible with 30 MHz or higher central frequency ultrasound imaging. The risk assessment for cap rupture is based on the relative size of the necrotic lipid pool, the presence of intramural evidence of blood, and on different features of the fibrotic cap, including the thickness and composition of the cap. The thinner the fibrotic cap, the higher the risk for cap rupture. That is, a loose and thin fibrotic cap has a high risk of rupture. A dense fibrosis with a thick cap means a low risk of rupture.

The risk for cap rupture is also related to the amount of macrophages and foam cells within the fibrotic cap. The presence of sonolucent lipid rich foam cells changes both the textural features (such as coarseness, business and complexity) and spectral features of the fibrotic cap. Ultrasound parameters derived from both texture analysis and the spectral analysis of the fibrotic cap may be included in the feature selection of vulnerable plaque in order to maximize the correct classification rate of a vulnerable plaque and the risk of plaque rupture.

In accordance with the invention, a relatively steep spectral slope characterizes dense fibrotic tissue with less risk for plaque rupture, while a relatively flat spectral slope characterizes moderate (less collagen) and loose fibrotic tissue, which are more vulnerable to plaque rupture. In one embodiment of the invention, the gradient of the spectral slope is characterized as follows. A spectral slope gradient of less than −0.3 dB/MHz is associated with dense fibrotic tissue, a spectral slope gradient ranging from −0.3 to −0.1 dB/MHz is associated with moderate fibrotic tissue, and a spectral slope gradient of less than −0.1 dB/MHz is associated with loose fibrotic tissue.

The density of the fibrotic cap is analyzed in accordance with the maximum power and mean power of the reflected ultrasound signal. Loose fibrotic tissue reflects less ultrasound energy (lower maximum and mean amplitude) than dense fibrotic tissue, as shown in FIG. 3. The density of the fibrotic cap can be characterized as follows. The average relative maximum from loose fibrotic tissue is approximately −20 dB, the average relative maximum for moderately loose fibrotic tissue is approximately −15 dB, and the average relative maximum for dense fibrotic tissue is less than approximately −10 dB. The average relative mean power from a loose fibrotic tissue is less than approximately 23 dB, the average relative mean power for moderately fibrotic tissue is approximately −20 dB, and the average relative mean power for dense fibrotic tissue is more than approximately −15 dB. With respect to the fibrotic cap, increased vulnerability for plaque rupture is thus based, but not limited, to the presence of a thin fibrotic cap (e.g., <100 um), spectral features for moderate to loose fibrotic tissue, and increased features for coarseness, entropy and complexity. These rules are incorporated into the fibrotic cap analysis module 54 as executable code in order to provide the user of the system 20 with information on the fibrotic cap.

The system 20 also includes a lipid core analysis module 56. The lipid core analysis module 56 incorporates rules to process the ultrasound data. In particular, the lipid core analysis module 56 identifies a nectrotic lipid core as a sonolucent region within the vessel wall. Maximum, minimum and average power of the reflected ultrasound signal from a tissue containing lipid is significantly less than from any fibrotic tissue (e.g., on average 5 dB). The lipid pool can be identified using low dynamic range settings (e.g., less than 40 dB) aimed towards the analysis of more sonolucent regions of the vulnerable plaque. The identification of the lipid pool can be improved by analyzing textural features like local uniformity (coarseness), contrast, and entropy of the lipid pool.

The size of the necrotic lipid core is directly related to the risk of plaque rupture—the more lipid a plaque contains, the higher the risk for plaque rupture. The size of the lipid pool can be calculated with respect to the total plaque area from both cross sectional images of the vessel wall and from a three-dimension re-construction of the vessel wall and lipid pool.

The presence of possible thrombus (already ruptured vulnerable plaque or intraplaque hemorrhage with no rupture on the fibrotic cap) can be identified using the thrombus analysis module 58. The presence of thrombus represents a high risk of rupture.

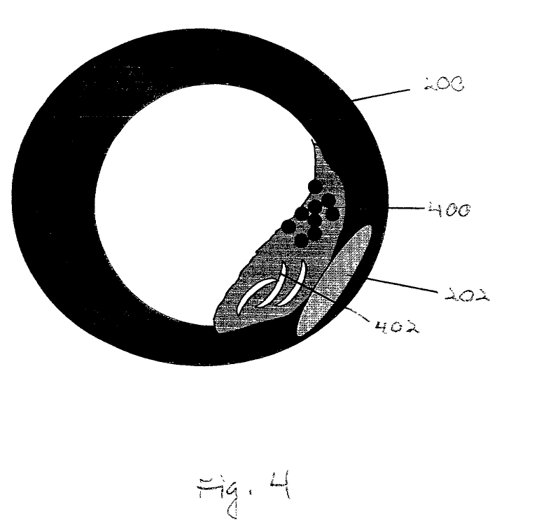

The thrombus analysis module 58 utilizes texture analysis of the back-scattered ultrasound signal. Thrombus, depending on the time of occurrence, can be either fresh (platelet rich) or older (red cell and fibrin rich), as shown in FIG. 4. In particular, FIG. 4 illustrates a normal vessel wall 200 and a lipid pool 202. The figure also illustrates a red cell rich region 400 and a fibrin rich region 402.

Red cells are relatively echo-reflective, but have specular characteristics that can be identified with texture analysis of the ultrasound signal. Red cell rich thrombus can therefore be identified with algorithms derived from first order statistics and with attributes of texture corresponding to spatial changes in intensity. Older thrombus, on the other hand, has a more heterogeneous appearance due to fibrin and plasma rich “lakes” within the platelets and red cells and can be seen as extremely low echogenic pools with typical textural features.

Intraplaque hemorrhage with no rupture on the fibrotic cap can also be used as one of the indicators for increased risk for plaque rupture, as red blood cells are very effective at transferring cholesterol to smooth muscle cells and macrophages and thus induce cellular inflammation and destabilize plaques.

A micro-calcification analysis module 60 is used to identify micro-calcification within the necrotic core. Micro-calcification may present a high risk of rupture. Micro-calcification is moderately echo-reflective (as moderately fibrotic tissue), but has characteristic specular features opposite to other similarly echo reflective components of a diseased vessel wall. Red cell rich thrombus has similar specular characteristics and spatial changes in intensity, but the maximum level of reflected ultrasound energy is significantly less (on average 8 dB) from thrombus than it is from micro-calcification. Post-mortem analyses of ruptured vulnerable plaques have shown that 70% of all ruptured plaques have evidence of plaque calcification, but convincing scientific evidence of its role as a risk factor for plaque rupture is still questionable. Therefore, the micro-calcification analysis module 60 reports the presence of micro-calcification and coarse calcium, the significance of which may be assessed by the attending physician.

The memory 40 also stores a vasa vasorum analysis module 62. The presence of vasa vasorum is often associated with vulnerable plaque and is believed to increase the risk for plaque rupture through capillary rupture leading to intramural hemorrhage and red cell invasion into the plaque. Due to the lack of previous animal model for vulnerable plaque no signal analysis techniques have been so far attempted to identify vasa vasorum. Although the identification of ruptured vasa vasorum is more important to assess the risk for vulnerable plaque rupture (intramural thrombus), the detection of vasa vasorum behind a lipid pool would further characterize the features of a vulnerable plaque. The vasa vasorum analysis module 62 analyzes the differences in backscattered power between adjacent regions behind the lipid pool and possible textural features aimed for the identification of branch like features extending towards the necrotic lipid core.

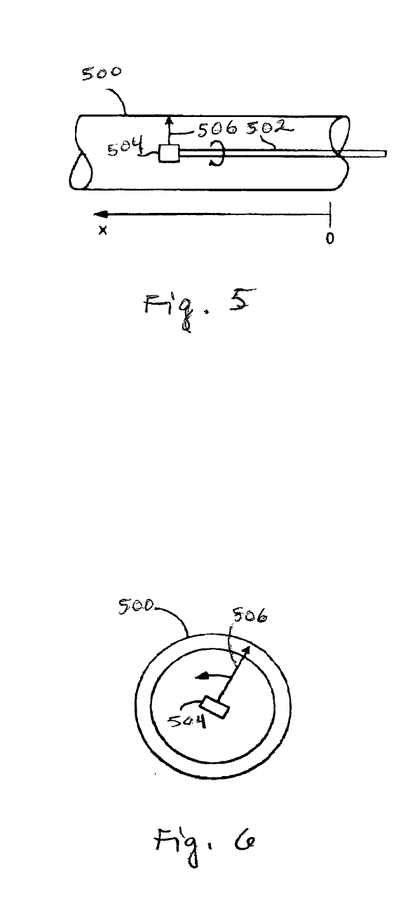

Although the invention has been fully described, the invention may be more fully appreciated in connection with the disclosure of alternate embodiments. FIG. 5 illustrates an ultrasound transducer 504 mounted on a catheter 502. The transducer 504 is introduced into an artery 500 of interest. Assume that the artery extends in an x-direction. The initial position (x=0) of the transducer is determined using any known method. As the transducer is rotated about the longitudinally extending, central x-axis of the artery, it is activated (transmit/receive) in order to generate a sequence of radial scan lines 506.

The echo return from each scan line is sensed and converted into a single echo power. The spectral analysis module 50 may be used to perform this operation. Although possible, it is not necessary to generate the scan lines as a conventional A-line, with many individual samples taken at different depths along the scan line; rather, for each scan line, a single ultrasound pulse can be generated, with the continuous echo profile being sensed. If multi-sample A-lines are used, however, their echo intensity values may be combined in any known manner to calculate a single power value. Because of the structure of the artery, time-gating will normally not be needed, although it may be used. All that is assumed is that some power value should be computed for each observed line, that is, for each angular position of the transducer.

In one embodiment of the invention, a full 360-degree annular section of the artery is scanned. In one implementation, 200 scan lines were generated with 1.8-degree angular separation. The number and separation of the scans lines can be selected differently; however, the optimum number and separation can be determined using normal experimental methods, taking into account the mechanical properties of the transducer and the apparatus that rotates it.

At each x-direction position, an annular section of the artery is therefore scanned with ultrasound, and a power value is generated for each scan line. According to the invention, the mean power of the ultrasonic echo signals for each annular section is then calculated. Assume, for example, that n (for example, n=200) scan lines are examined at each transducer position x, and that the echo power of each scan line is p(x,i) (i=1, . . . , n). The mean power value P(x) at position x can therefore be calculated as follows:

$$P ( x ) = \frac{1}{n} {\sum }_{i = 1}^{n} P ( x , i )$$

The mean power value is preferably normalized. In the preferred embodiment of the invention, the transducer is calibrated by determining the echo signal power W received from a perfectly specular reflector. Such calibration is known in the art. The calibration and normalization methods used in an embodiment of the invention are as described in Spencer, et al., “Characterisation of Artherosclerotic Plaque By Spectral Analysis of Intravascular Ultrasound: An In Vitro Methodology,” Ultrasound in Med. & Biol., Vol, 23, No. 2, pp, 191-203, 1997. At each position, the mean power value P(x) is therefore calculated as follows:

$$P ( x ) = \frac{k}{w} \cdot \frac{1}{n} {\sum }_{i = 1}^{n} P ( x , i )$$

where k is an optional scaling factor, which may be chosen, for example, to ensure that all values fall within a desired range for convenient display. Using the normalization method described in the Spencer paper, mean power is expressed in decibels. Of course, other known normalization methods may also be used.

The transducer is then moved by a known amount to a new position within the artery, for example, by pulling it using a precision motor that moves an arm to which the catheter is connected. In one implementation, the transducer is moved in 200 μm increments (Δx=200 μm). Another annular scan is then performed and a new mean power value is then obtained at the new position. The transducer is then moved again, and so on, until the entire length (from x=0 to some final position x₁ of interest of the artery is scanned). At that point, there will be x₁/Δx normalized mean power values P(x), each representing the normalized mean power returned from one annular section of the scanned artery.

According to an embodiment of the invention, the mean power values are examined and used to determine the presence of vulnerable plaque, in particular, of a fibrotic cap and a lipid pool. Note that the extent of development of these two structures strongly correlates with the risk of rupture of the artery due to the vulnerable plaque. The following ranges of normalized mean power values P(x) indicate the presence of the following structures at each position of the artery:

| P(x) range | Mid-Range | |
|---|---|---|
| (db) | P(x) Value | Structure |
| −18 to −30 | −24 | Lipid pool |
| −15 to −9 | −12 | Fibrotic cap - Moderate fibrosis |
| −9 to −3 | −6 | Fibrotic cap - Dense fibrosis |

A clinician can then examine the normalized power values obtained in the actual scan, compare them with the ranges above, and identify any scanned section of the artery whose normalized mean power value indicates, for example, a fibrotic cap or a lipid pool. Note that a dense and thick cap fibrotic cap tends to indicate a low risk of rupture, whereas a moderate and thin cap means high risk of rupture. The normalized power values may also be processed by the fibrotic cap analysis module 54, which provides an indication of a fibrotic cap of moderate fibrosis or dense fibrosis based upon the ranges set forth above. The normalized power values may also be processed by the lipid core analysis module 56, which provides an indication of a lipid pool based upon the ranges set forth above.

A characteristic, normalized mean power range may also be developed for other structures. A thrombus, for example, has normalized mean power of −15±2. This represents a slight overlap with moderate fibrosis but is identifiable as a “lake” within the lipid pool as opposed to a cap over the lipid pool. The thrombus analysis module 58 may be used to apply the foregoing criterion that identifies thrombus.

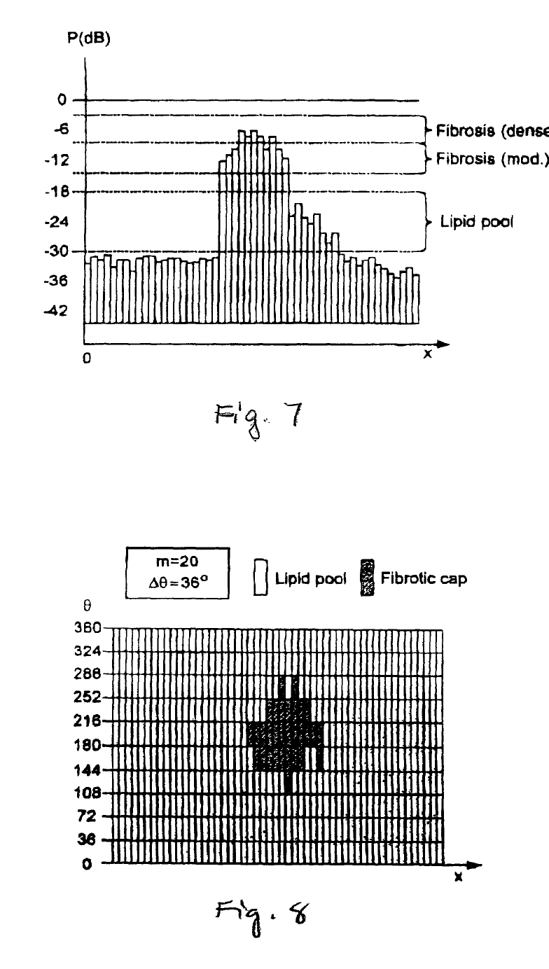

The data-rendering module 53 may be used to graphically display the normalized mean power values. FIG. 7 illustrates a mean power display graph, in which mean power P(x) values are displayed as a function of position x. Guide bands indicating, for example, a lipid pool range, a moderate fibrosis range, and a dense fibrosis range, can then be displayed as an overlay to aide in interpreting the power values. The power values may also be automatically processed using the various modules stored in memory 40. For example, the lipid core analysis module 56 may be used to identify the lipid pool range.

The display of power values can also be color-coded. For example, normalized mean power values that correspond to structures indicative of vulnerable plaque (such as the fibrotic cap and lipid pool) can then be displayed with easy-to-see colors, such as red and yellow. The graph shown in FIG. 7 could also be color-coded.

As is mentioned above, a full 360-degree scan may be performed at each transducer position. Vulnerable plaque will typically not extend for a full 360 degrees. Consequently, it is not necessary to calculate a single normalized power value for the entire 360-degree scan annulus. Rather, the echo power values for m scan lines could be grouped so as to correspond to angular sectors of Δθ degrees of arc. At each transducer position x, there would then be m=360/Δθ groups, each containing values from n/m scan lines. Assuming as above, for example, that n=200, then one could have ten groups of m=20 scan lines, each group corresponding to a 36-degree sector.

The system can then calculate and display a normalized mean power value for each group, for each transducer position x. Each of these values can then be displayed with color-coding. FIG. 8 illustrates this alternative, where, by way of example, the mean power values indicative of a fibrotic cap are located mostly in the angular range of 144-252 degrees, and the lipid pool lies mostly in the angular range of 180-252 degrees. The number m, and thus Δθ (the angular size of groups), could be made user-adjustable, with the display being updated accordingly. Note that m=1 corresponds to the case above, with a single normalized mean power value for an entire 360-degree annular sector at each transducer position. By adjusting the value of m, the clinician can then see a varying display with varying resolution.

In most practical applications it will not be necessary for the clinician to know exactly what the angular position of the transducer is, even where more than one scan line group is displayed for each position x. Rather, a display as in FIG. 8 will simply help the clinician to obtain a better idea of the angular extent of the vulnerable plaque.

In one working prototype of the invention, the numerical ranges indicating different plaque structures were determined as follows. Several portions of arteries taken from fresh cadavers were mounted in a bracket, in a saline solution maintained at approximately a normal blood pressure of 80 mmHg. A calibrated ultrasound transducer was then introduced into each arterial portion, which was then scanned as described above, that is, as 360-degree annular sections at different positions (at 200 μm increments) in the x-direction, over an entire predetermined length of the arterial portion. The transducer was withdrawn at 200 μm increments using a precision stepper motor.

Each arterial portion (whose absolute position in the x-direction was known from the bracketing arrangement) was then sectioned and examined visually by a pathologist under a microscope. The normalized mean power values were then compared with the pathologist's visual determination. The normalized mean power value ranges tabulated above had a high degree of correlation with the pathologist's findings of the presence of lipid pools, fibrotic caps, etc.

The foregoing description, for purposes of explanation, used specific nomenclature to provide a through understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, the thereby enable other skilled in the art to best utilize the invention and various embodiments with various modifications as are suited to the particular use contemplated. It is intended that the scope of the invention be defined by the following claims and their equivalents.

In the claims:

1. A method of ultrasonically identifying vulnerable plaque, comprising:
gathering an intra-vascular ultrasound data signal;
characterizing said intra-vascular ultrasound data signal as a function of relative amplitude and frequency to define a spectral slope associated with fibrotic tissue; and
identifying vulnerable plaque based upon said spectral slope.

2. The method of claim 1 further comprising identifying vulnerable plaque based upon a texture analysis of said intra-vascular ultrasound data signal.

3. The method of claim 1 further comprising locating a fibrotic cap based upon said spectral slope.

4. The method of claim 1 further comprising locating a lipid core using a low dynamic range setting to facilitate the identification of a sonolucent region of vulnerable plaque.

5. The method of claim 1 further comprising locating thrombus through a texture analysis of back scattered intra-vascular ultrasound data.

6. The method of claim 1 further comprising locating micro-calcification of a necrotic core by distinguishing specular features of echo-reflective intra-vascular ultrasound data.

7. The method of claim 1 further comprising locating vasa vasorum by identifying branch like features in back scattered intra-vascular ultrasound data.

8. A method of ultrasonically identifying vulnerable plaque, comprising:
gathering an intra-vascular ultrasound data signal;
characterizing said intra-vascular ultrasound data signal as a mean power signal; and
identifying vulnerable plaque based upon said mean power signal.

9. The method of claim 8 wherein identifying includes identifying a nectrotic, lipid pool based upon said mean power signal, said method further comprising characterizing the risk of vulnerable plaque rupture based upon the size of said necrotic, lipid pool.

10. The method of claim 8 wherein identifying includes identifying a fibrotic cap based upon said mean power signal, said method further comprising characterizing the risk of vulnerable plaque rupture based upon the density and thickness of said fibrotic cap.

11. The method of claim 8 wherein identifying includes identifying thrombus based upon said mean power signal, said method further comprising assigning a risk of vulnerable plaque rupture based upon the presence of said thrombus.

12. The method of claim 8 wherein identifying includes identifying vasa vasorum based upon said mean power signal, said method further comprising assigning a risk of vulnerable plaque rupture based upon the presence of said vasa vasorum.

13. The method of claim 8 wherein identifying includes identifying micro-calcification based upon said mean power signal, said method further comprising assigning a risk of vulnerable plaque rupture based upon the presence of said micro-calcification.

14. The method of claim 8 further comprising displaying said mean power signal as a mean power display graph.

15. The method of claim 14 further comprising superimposing a lipid pool range on said mean power display graph.

16. The method of claim 14 further comprising superimposing a dense fibrosis range on said mean power display graph.

17. The method of claim 14 further comprising superimposing a moderate density fibrosis range on said mean power display graph.

18. The method of claim 14 further comprising displaying said mean power signal as a function of ultrasound scan angle.
